(19) United States
(12) Patent Application Publication
(10) Pub. No.: US 2004/0260152 A1
(43) Pub. Date: Dec. 23, 2004

(54) Speculum assembly

(76) Inventors: Jeanene Sant, Preston, ID (US); Howard M. Tanner, Logan, UT (US)

Correspondence Address: COLLIER SHANNON SCOTT, PLLC, 3050 K STREET, NW, SUITE 400, WASHINGTON, DC, 20007, US

(21) Appl. No.: 10/465,641

(22) Filed: Jun. 20, 2003

(51) Int. Cl. A61B 1/32

(52) U.S. Cl. 600/184

(57) ABSTRACT

The present invention is directed to a speculum assembly for single-use in colon lavage therapy. The speculum assembly comprises a tubular body to which a tubing-set is attached. The tubular body has a leading end, a middle portion, and a trailing end. The leading end has a tapering body transitioning into a tip, a scallop opening adjacent to the tip, and at least one annular rib about the tubular body, positioned between the scallop opening and the middle portion. The middle portion has a flange. The flange is in communication with a portion of an irrigation tube. The trailing end of the tubular body comprises a handle that selectively retains a portion of the irrigation tube and attaches to a portion of a waste hose, elements of the tubing set.

### FIELD OF THE INVENTION

The present invention relates to a speculum and in particular, to a rectal speculum for use with lavage systems.

### BACKGROUND

The cleansing of the large intestine and specifically, the colon by lavage techniques is well documented. At the ileocecal valve, partially digested material passes from the small to large intestine and thereafter, through the ascending, transverse, and descending colon to the rectum where it is eliminated. The corrugated, sacculated wall of the colon frequently traps exiting waste material that can subsequently become a source for infection, e.g., colitis, constipation, irritable bowel and occasionally, the nidus for disease processes, e.g., Chrohn's disease, heart burn, and diverticulitis.

Lavaging of the colon commonly involves the insertion of a speculum, a tapered hollow tube, into the rectum to a precise position within the pelvic colon. The smaller tube end is positioned within the colon while the larger is attached, outside the body, to a tube through which the trapped waste products are monitored while being discharged. A secondary tube, which is attached to an inlet incorporated within the speculum, enables delivery of irrigation fluid to the colon under appropriate control and the further fluidization of exiting waste.

There are numerous prior-art specula that with varying degrees of success, aid the removal of trapped waste products from within the colon. In particular, U.S. Pat. No. 4,712,536 (“the '536 patent”) to Hawks, issued Dec. 15, 1987, describes a speculum having a concentric tube construction. The concentric tube has an inner tube (obturator) having a distal detailing to aid in-patient placement, and is removably located within an outer tube (speculum). Following the placement of the speculum assembly within the colon, the obturator is removed from within its core. Thereafter, the potentially unsanitary (because exiting fecal matter and associated fumes will escape), attachment of a drain hose to the speculum's proximal end is affected. The speculum incorporates a proximally positioned and facing tubular inlet that enables both the initial filling of the colon with irrigation fluid and subsequent, dilution and flushing of waste material. A thumb rest is positioned diametrically opposite the tubular inlet, which aids the mid-procedure attachment of the drain hose.

Subsequent to the '536 patent, U.S. Pat. No. 4,842,580 (“the '580 patent”) to Ouelette issued Jun. 27, 1989. The '580 patent describes a speculum that is substantially similar to the '536 patent in construction and functioning. The fluid inlet of this design, however, enters the speculum at an intermediate point along its length. Additionally, a circular flange, providing a uni-positional stop, abuts the inlet and fluid passing there through is directed distally towards the colon. The inlet's positioning, however, encumbers both the therapy and the therapist's comfort.

In addition, U.S. Pat. No. 4,874,363 (“the '363 patent”) to Abell, issued Oct. 17, 1989, describes a non-obturator speculum comprising, in a forward half, a distally tapering tube that terminates in a closed tip feature into which two media apertures are formed. The tube's flared proximal detailing assists the products in-treatment retention. The fluid inlet of this design is transversely oriented with respect to the speculum, located at an intermediate position along the product's length, and problematically, radially aligned with the central axis of the media apertures. When the media apertures are positioned for optimum performance, the inlet hose will press unnecessarily into the patient's buttocks. Conversely, when the inlet hose is appropriately positioned between the patient's buttocks, the media apertures will be substantially occluded by the colon's anatomy.

U.S. Pat. No. 4,943,285 (“the '285 patent”) to Hawks, issued Jul. 24, 1990, describes an enhancement to the '536 patent. As in the '363 patent, the speculum, a concentric tube, incorporates an in-patient retention profile that comprises annular ribs, between which the sphincter muscle is temporarily trapped during a treatment. Because every patient has a unique anatomy and physiology, the ‘fixed’ retention feature will function in some and will be ‘ejected’ by others, thus, rendering it completely unreliable.

U.S. Pat. No. 5,176,630 (“the '630 patent”) to Shilling, issued Jan. 5, 1993, describes a non-obturator speculum having a distally tapered tube in a forward half that terminates in a closed tip feature into which at least one media aperture is formed, and detailing within its proximal, rear half that provides fluid pulsatility. As in the '285 patent, the speculum incorporates an in-patient retention feature in the form of an inflatable cuff, the feature's performance remains patient-dependant. As the '580 patent, the '630 patent provides a uni-positional stop on its forward half. The geometric relationship between the media aperture(s) and the fluid inlet, as in the '363 patent, is contradictory.

U.S. Pat. No. 5,351,674 (“the '674 patent”) to Hawks, issued Oct. 4, 1994, describes a speculum that is substantially similar in conception to the '363 patent that is of unitary construction. The '674 patent replicates the distal features and design of the '630 patent but discusses the potential aid afforded by the form of a distal tip to the intestine in prolapsus. The aid, however, is withheld as a consequence of the geometry. Similar to the '363 patent, optimum positioning of the speculum to support the transverse colon, while affording the inlet hose, an appropriate between-buttocks positioning, will obscure the media aperture(s) in the colon's anatomy. Conversely, if the speculum is positioned for optimum removal of waste, the prolapsed colon will obscure the media aperture(s) and the inlet hose will press unnecessarily into the patient's buttocks. The speculum shown in U.S. Patent No. Des. 385,628 is predicated upon the '674 patent and differs only in the specific detailing of the tubular inlet.

The speculum described in U.S. Pat. No. 5,931,776 (“the '776 patent”) to Dotolo, issued Aug. 3, 1999, has a dissolvable tip at its distal end. The '766 patent is a hybridization of the prior art with a single piece tubular body that at its proximal end accepts a drain hose and a distally directed inlet hose. With use and the timely dissolution of the tip, the product presents and functions as the original obturator assisted device of the '536 patent. This, however, is predicated upon the ‘timely’ dissolution of the tip.

It is an advantage of some, but not necessarily all, embodiments of the present invention to provide a specific geometric relationship between the media aperture(s) and the fluid inlet that conforms to the anatomical and procedural realities associated with colonic lavage.

It is a further advantage of some, but not necessarily all, embodiments of the present invention to incorporate a distal tip that maximizes both patient comfort and product performance while maintaining the product's structural integrity.

It is yet another advantage of some, but not necessarily all, embodiments of the present invention to optimize the fluid inlet positioning within the speculum, while concurrently maintaining therapist convenience and comfort during its use.

It is yet another advantage of some, but not necessarily all, embodiments of the present invention to incorporate the ‘Venturi’ principle in the delivery of irrigation fluid to the speculum to aid flushing of the main treatment conduit.

It is yet another advantage of some, but not necessarily all, embodiments of the present invention to incorporate detailing that will promote the re-toning of the sphincter muscle, and diminish the patient's reliance upon lavage, unlike existing products that codify an unchanging relationship between patient and procedure.

It is still another advantage of some, but not necessarily all, embodiments of the present invention to integrate the fluid supply hose, waste hose, and speculum into a sanitary, single use disposable speculum assembly.

It is still another advantage of some, but not necessarily all, embodiments of the present invention to provide an adjustable positioner that will enable the speculum's individualized, in-colon, in-treatment positioning.

Additional advantages of various embodiments of the invention are set forth, in part, in the description that follows and, in part, will be apparent to one of ordinary skill in the art from the description and/or from the practice of the invention.

### SUMMARY

Responsive to the foregoing challenges, Applicant has developed a novel speculum assembly. According to at least one embodiment of the invention, a speculum assembly for single-use in colon lavage therapy comprises: a tubular body having a leading end, a middle portion, and a trailing end; the leading end has a tapering body transitioning into a tip, a scallop opening adjacent to the tip, and at least one annular rib about the tubular body, positioned between the scallop opening and the middle portion; the middle portion has a flange, the flange is in communication with a portion of an irrigation tube; and the trailing end of the tubular body comprises a handle that selectively retains a portion of the irrigation tube, and attaches to a portion of a waste hose.

According at least another embodiment of the invention, the speculum assembly for single-use in colon lavage therapy comprises: a tubular body having a leading end, a middle portion, and a trailing end; the leading end has a tapering body transitioning to a tip, a scallop opening adjacent to the tip, and at least one annular rib about the tubular body, disposed between the scallop opening and the middle portion; the middle portion incorporates a detachable adjustable positioner and a mechanism for adjustments along the tubular body, the adjustable positioner has at least one flexible tab; and the trailing end comprises a handle, the handle is adapted to accept a portion of an irrigation tube and the trailing end selectively retains a portion of a waste hose.

According to at least yet another embodiment of the present invention, the speculum assembly for single-use colon lavage therapy comprises: a first tubular body having a tapered sleeve comprising a tip, a scallop opening, an annular rib about the first tubular body, and a reduced diameter band; and a second tubular body to which the first tubular body is assembled, the second tubular body has a leading end, a middle portion and a trailing end; the leading end is complimentary to the first tubular body; the middle portion incorporates a mechanism that corresponds with an adjustable positioner assembled thereon, enabling positional adjustment along the second tubular body, the adjustable positioner incorporates at least one flexible tab; and the trailing end comprises a handle, the handle is adapted to accept a portion of an irrigation tube and a portion of a waste hose.

According to at least another further embodiment of the present invention, the speculum assembly for single-use colon lavage therapy comprises: a first tubular tapered sleeve comprising a tip, a scallop opening, and a flange, the flange has a snap-groove incorporated therein; and a second tubular body is snap-fitted to the snap-groove of the first tubular body, the second tubular body is complimentary to the first tubular body having an annular snap-ring about a middle portion and comprises a handle, the handle is adapted to accept a portion of an irrigation tube and a portion of a waste hose.

### BRIEF DESCRIPTION OF THE DRAWINGS

In order to assist the understanding of this invention, reference will now be made to the appended drawings, in which like reference characters refer to like elements.

FIG. 1 is a perspective view of an embodiment of a speculum assembly.

FIGS. 2A and 2B are perspective views of a leading end of the speculum assembly.

FIG. 3 is a perspective view of the leading end with one rib, which rests on the sphincter muscle during a treatment and, a scalloped opening.

FIG. 4 is an exploded view of the speculum assembly, in particular, an embodiment of the elbow component.

FIG. 5 is a longitudinal cross-section of an embodiment of the speculum assembly's tubular body.

FIG. 6 is a perspective view of a further embodiment of the speculum assembly.

FIG. 7 is a perspective view of the speculum assembly illustrated in FIG. 6.

FIG. 8 is a perspective view of a further embodiment of the speculum assembly.

FIG. 9 is a perspective view of a further embodiment of the speculum assembly.

FIG. 10 is an exploded perspective view of the speculum assembly illustrated in FIG. 9.

### DETAILED DESCRIPTION OF EMBODIMENTS OF THE INVENTION

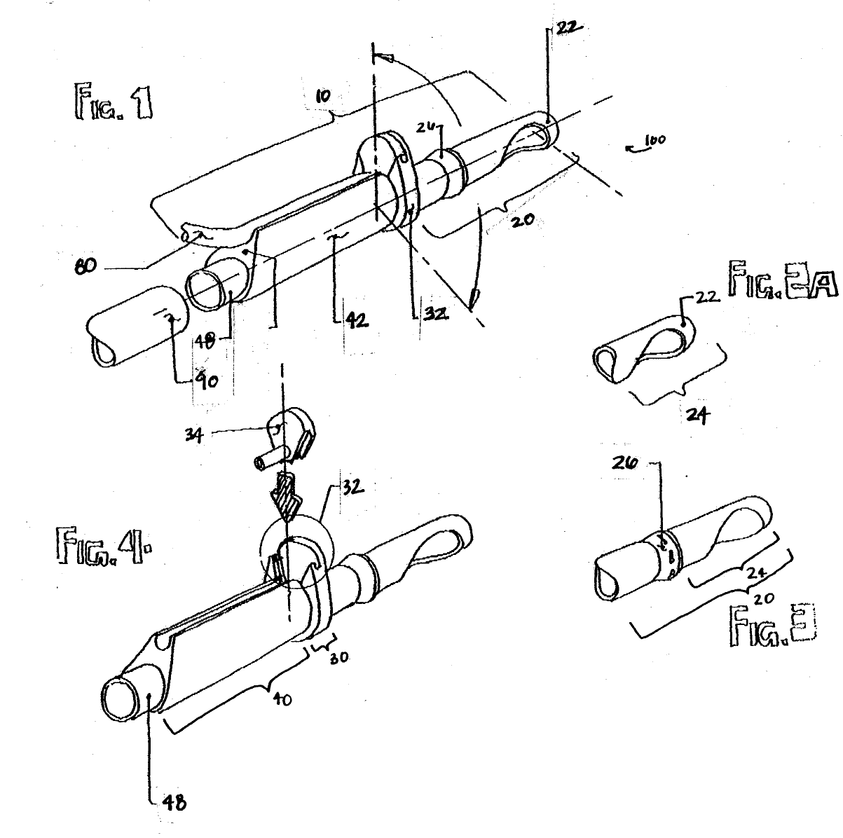

Reference will now be made in detail to embodiments of the present invention, an example of which is illustrated in the accompanying drawings. With reference to FIG. 1, an example embodiment of a speculum assembly 100 is illustrated. The speculum assembly 100 comprises a hollow tubular body 10 having a leading end 20, a middle portion 30, and a trailing end 40. In FIG. 1, the speculum assembly may be, but is not limited to, a unitary construction. The leading end 20 represents the treatment end of the assembly that is positioned within a patient's colon during lavage. The trailing end 40, however, is positioned outside of the patient's body, and the middle portion 30 limits the position of the leading end 20.

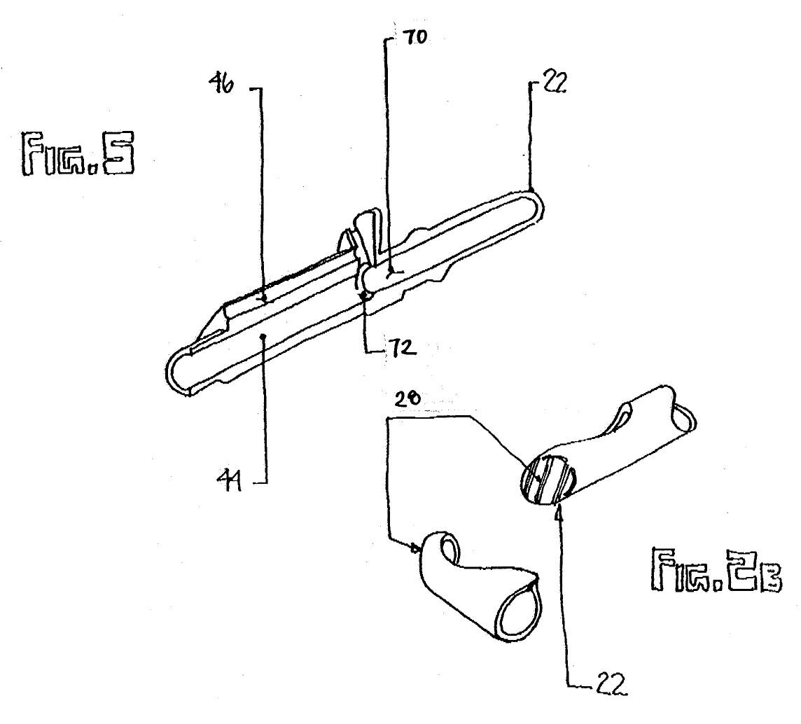

In FIG. 1, the leading end 20 of the tubular body 10 may comprise a tip 22, a scallop opening 24, and at least one rib 26. In FIG. 3, the leading end 20 of the speculum assembly 100 transitions smoothly to a hemispherically shaped tip 22. The shape of tip 22 is not limited to a tapered, a rounded, or similar configuration, but to a configuration that minimizes the possibility of patient discomfort or injury to the colon or intestinal structures during insertion of the speculum assembly 100. In some embodiments, the hemispherical tip 22 may, but is not limited to, incorporate an oblique facet 28 as FIG. 2B.

In FIG. 2A, the scallop opening 24 is shown located proximal to the tip 22. The scallop opening 24 affords a maximized unrestricted irrigation of the colon, minimized restriction of waste removal there from, and does not affect the speculum assembly's structural integrity. In some example embodiments, the scallop opening 24 is a curved notch, an orifice, or any other opening into the hollow tubular body 10. Embodied in FIG. 2A, the scallop opening 24 is a curved notch having a generally ob-round shape with a smooth edged feature.

The rib 26 found on the leading end 20 is located between the scallop opening 24 and the middle portion 30 of the assembly 100. The rib 26 may stimulate the therapeutic contraction and relaxation of the sphincter muscle when resting thereupon during a patient treatment. As in FIGS. 1 and 3, the rib 24 may be, but is not limited to, a triangulated configuration that circumscribes the tubular body 10 or may be any other configuration stimulating the therapeutic contraction and relaxation of the sphincter muscle.

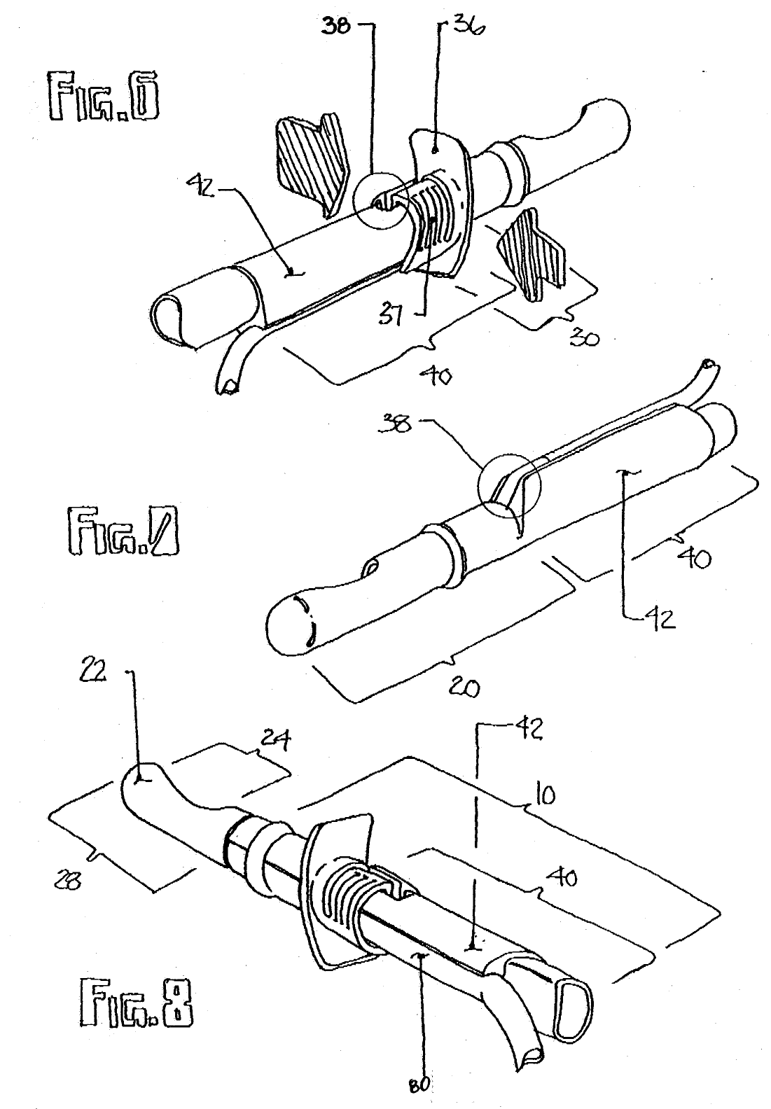

The middle portion 30 of the speculum 100 may include a flange 32 located at an intermediate point along the tubular body 10, as shown in FIGS. 1 and 4. In some embodiments, the flange 32 may be detailed to accept an elbow component 34, as illustrated in FIG. 4. In further embodiments, the flange 32 may be positionally adapted for each patient's unique anatomy. The flange 32 is detailed to mimic or correspond to the geometry of the surrounding anatomy. The flange 32 protrudes from the tubular body 10 at an intermediate point along its length and may be radially rotated with respect to the scalloped opening 24, as shown in FIG. 1. The flange 32 may also define a point of transition of a fluid irrigation tube 80 and a main conduit 44, as shown in FIGS. 1 and 5.

In some example embodiments, the trailing end 40 of the tubular body 10 comprises a handle component 42 positioned proximal to the flange 32 of the middle portion 30. The handle 42 has, but is not limited to, an ovoid profile. The sectional view in FIG. 5 identifies the major lumen 44 that extends distally to a radiussed fillet 72 and a counterbore 70 corresponding with the irrigation tube 80 at the elbow 34 and extending there from to a point adjacent to the tip 22, and a minor lumen 46 that shares the profile of the handle 42. The main conduit 44 is a waste lumen of the assembly 100 and the minor lumen 46, is an inscribed recess to accommodate an irrigation tube 80. The irrigation tube 80 may be connected to the elbow component 34 assembled within the recess 46 thereby coalescing smoothly within the profile of handle 42.

In FIG. 4, the elbow component 34 may be adapted to the flange 32 such that the elbow 34 and the flange 32 are secured creating a unitary configuration. The elbow and flange may be bonded, fused, snap-fifted to one another, or secured together by any other appropriate means. The elbow component 34 embodied in FIG. 4, having a fluid passageway there through, is not limited to this configuration, and may be configured to create a secure fit with the recess 46. The elbow and the recess may be bonded, fused, snap-fitted to one another, or secured together by any other appropriate means. The elbow component 34 may also comprise an 0-ring seal at its junction with the tubular body 10 at the recess 44. The flange 32 and the elbow component 34 potentially incorporated therein, facilitate the enhanced cleansing of the main conduit 44 by incorporating a flow restriction that results in velocity and pressure changes of fluid in accordance with the Venturi principle. In an embodiment, the proximal exiting waste material passes through the assembly's main conduit 44 to flow across the distal opening of the elbow component 34 and is thereby further fluidized by irrigation fluid, which is drawn in a Venturi-manner into the main conduit 44 at a radiussed fillet 72.

The proximal end of the trailing end 40, as shown in FIG. 1, terminates in a boss 48. The boss 48 is in communication with a waste hose 90. The waste hose 90 may be retained on or connected to the boss 48 by a rib, a barb, a tapering detail, or any other suitable means inseparably linking the boss 48 to the waste hose 90. In another example embodiment illustrated in FIGS. 6 and 7, the speculum assembly 100 is modified to incorporate an adjustable positioner 36 that enables control of the depth to which the speculum may penetrate the pelvic colon of each individual patient. The middle portion 30 and the flange 32, of FIGS. 1 through 5, are modified to accommodate an adjustable positioner 36. In particular, the handle component 42 of the trailing end 40 transitions into the middle portion 30, which may include a stepped feature 38.

The adjustable positioner 36 is slidably positioned over the handle component 42. The adjustable positioner's 36 detailing is configured to compliment the handle's exterior profile. In some embodiments, the adjustable positioner may be detachable from the tubular body 10. In FIG. 7, the handle component 42 of the trailing end 40 and the leading end 20 transition into the middle portion 30 along the tubular body 10 toward the adjustable positioner 36 with the step feature 38. Instead of the recess 46 of the trailing end 40 terminating at the flange 32 illustrated in FIGS. 1 through 5, the recess 46 extends to the step feature 38. In some but not all embodiments, the step feature 38, a molded-in ratchet mechanism, may be positioned directly opposite the recess 46. The adjustable positioner 36 incorporates a plurality of flexible tabs 37 having outwardly facing pawl details, which correspond to the molded-in ratchet 38 within the handle 42. When the tabs 37 are squeezed together their pawl detailing disengages from the ratchet 38 thereby enabling the positioner's 36 adjustment along the tubular body 10. The ratchet-and-pawl based adjustment detailing of this embodiment may be replaced by rack-and-pinion, a canted plate having a hole there through corresponding with the handle profile 42, or any similar mechanism for adjustments (linear and/or vertical) along the tubular body.

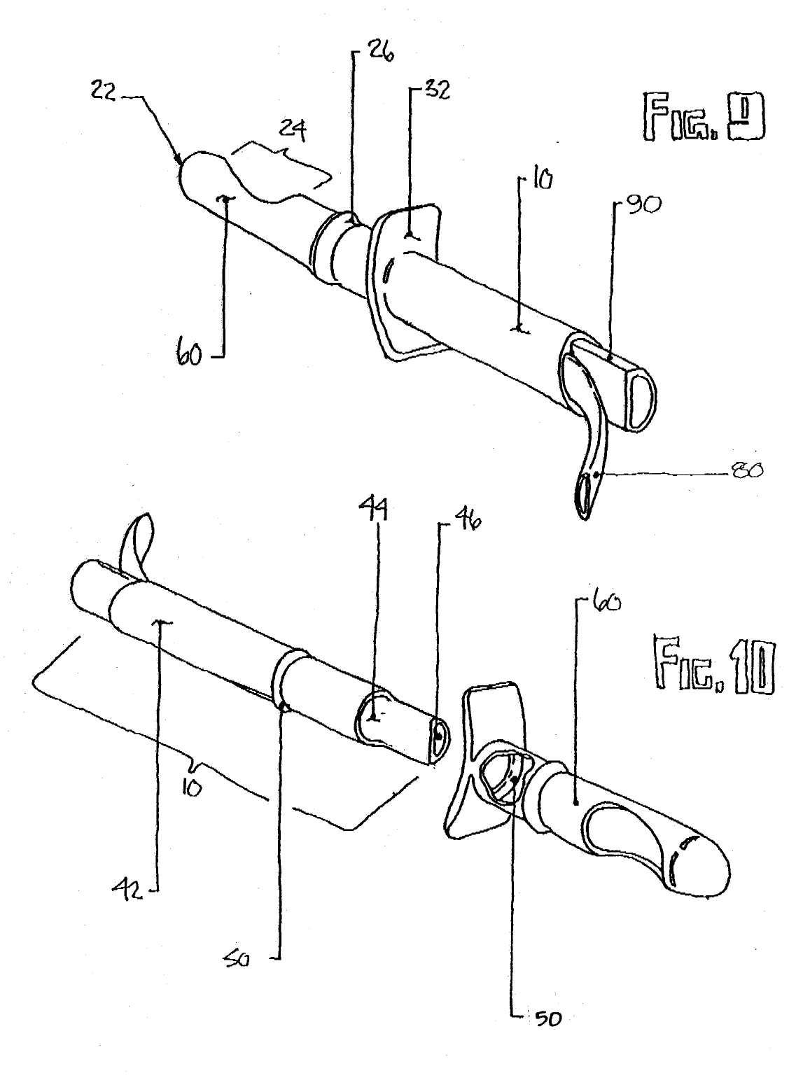

In an additional example embodiment shown in FIG. 8, the speculum assembly 100 is modified to include an irrigation tube 80, which traverses the speculum assembly's entire length and/or a substantial portion as illustrated in FIG. 8. This enhances the colon's irrigation, fluidizes the exiting waste, and cleanses the conduit. In particular, the ovoid profile of the handle component 42 extends from the speculum's trailing end 40 and tapers distally, terminating at a point immediately proximal of the scallop opening 24. The tip 22 and the scallop opening 24 of the preceding embodiments are incorporated here into a unitary tip component, which is permanently assembled to the tubular body 10.

In yet another example embodiment illustrated in FIG. 9, the speculum assembly 100 comprises at least two tubular bodies. The first tubular body is a tapered sleeve 60. The tapered sleeve 60 may include the tip 22, the scallop opening 24, the rib 26, and the flange 32 of the speculum assembly illustrated in FIGS. 1 through 5, and their attendant functional benefits. In some further embodiments, the tapered sleeve 60 may include a reduced diameter band ensuring connection with a second tubular body 10, a snap-ring, or any other means necessary to connect the first and second bodies together. In other embodiments, the flange 32 may include a snap-groove 50. The snap-groove 50 may be, but is not limited to, a semi-circular configuration that enables a snap fitting of the first tubular body 60 to the second tubular body 10.

The second tubular body 10 may have two lumen passing there through, a waste lumen 44 and an irrigation tube 46, and incorporates a handle 42. The waste lumen 44, the irrigation tube 46, and the handle 42 illustrated in FIG. 10 comprise all the attendant functional benefits as described above with reference to FIGS. 1 through 8. In some embodiments, the second tubular body 10 may have a leading end, a middle portion, and a trailing end. The leading end is complementary to the tapered sleeve 60 within which it is positioned. The middle portion incorporates a mechanism for corresponding with the adjustable positioner 36. The mechanism may be, but is not limited to, a molded-in ratchet, a rack-and-pinion, a canted plate having a hole there through corresponding wit the handle 42, or any other mechanism for adjustments (linear and/or vertical) along the tubular body. The trailing end has a handle 42, which is adapted to accept a portion of the irrigation tube 80 and a portion of the waste hose 90.

In FIG. 10, the second tubular body 10, is separated from the first tubular body, the tapered sleeve 60. The proximal end of a tapered sleeve 60 incorporates a flange 32 that aids the assembly's in-treatment positioning.

The speculum assembly 100 may be composed of, but is not limited to, biocompatible materials that withstand sterilization e.g., medical-grade polypropylene, polyethylene, acrylonitrile butadiene styrene (ABS), or any other suitable material. The irrigation tube and the waste tube may be composed of, but not limited to, polyethylene or similar material.

The speculum assembly 100 comprises a group of related molded components to which a tubing set is permanently attached. The tubing set comprises a waste hose and an irrigation tube which are configured proximally to attach to the assembly, and distally, to detachably connect to a control module. The control module regulates the volume and rate at which irrigation fluid is delivered to the assembly. The control module also monitors exiting waste and its discharge into an appropriate receptacle. The control module has hardware that enables the therapist's routine performance of colon lavage.

Numerous characteristics and advantages have been set forth in the foregoing description, together with details of structure and function. The novel features are pointed out in the appended claims. The disclosure, however, is illustrative only, and changes may be made in detail, especially in matters of shape, size, and arrangement of parts, within the principle of the invention, to the full extent indicated by the broad general meaning of the terms in which the appended claims are expressed.

What is claimed is:

1. A speculum assembly for single-use in colon lavage therapy comprising:
a tubular body having a leading end, a middle portion, and a trailing end; the leading end has a tapering body transitioning into a tip, a scallop opening adjacent to the tip, and at least one annular rib about the tubular body, positioned between the scallop opening and the middle portion; the middle portion has a flange, the flange is in communication with a portion of an irrigation tube; and the trailing end of the tubular body comprises a handle that selectively retains a portion of the irrigation tube and attaches to a portion of a waste hose.

2. The assembly of claim 1, wherein the irrigation tube and the waste hose are components of a tubing set permanently attached to the speculum and selectively attached to a control module.

3. The assembly of claim 1, wherein the assembly is of unitary construction.

4. The assembly of claim 1, wherein the flange and the scallop opening are radially offset from one another about a longitudinal axis of the tubular body.

5. The assembly of claim 1, further comprising a boss, the boss is adapted to the trailing end, wherein the boss connects the tubular body of the speculum assembly to the waste hose.

6. The assembly of claim 5, wherein the tubular body further comprises at least two lumen, a waste lumen extending between the tip and the boss and an inscribed recess extending from the middle portion to the trailing end, wherein the recess is adapted to receive the irrigation tube.

7. The assembly of claim 6, further comprising an elbow component having a fluid passageway there through, the elbow component is in communication with the flange, wherein the elbow component and the flange are adapted to receive the irrigation tube when positioned within the recess of the tubular body.

8. The assembly of claim 7, wherein the elbow component incorporates at least one Venturi-type restrictor through which irrigation fluid flows into the waste lumen of the tubular body.

9. The assembly of claim 1, wherein the flange corresponds to an individual patient.

10. The assembly of claim 1, wherein the flange is an adjustable positioner, the adjustable positioner is selectively located along the tubular body to accommodate an individual patient.

11. The assembly of claim 1, wherein the handle has an ovoid configuration.

12. The assembly of claim 1, wherein the rib has a triangular configuration.

13. A speculum assembly for single-use in a colon lavage therapy comprising:
a tubular body having a leading end, a middle portion, and a trailing end; the leading end has a tapering body transitioning to a tip, a scallop opening adjacent to the tip, and at least one annular rib about the tubular body, disposed between the scallop opening and the middle portion; the middle portion incorporates a detachable adjustable positioner and a mechanism for adjustments along the tubular body, the adjustable positioner has at least one flexible tab; and the trailing end comprises a handle, the handle is adapted to accept a portion of an irrigation tube and the trailing end selectively retains a portion of a waste hose.

14. The assembly of claim 13, wherein the mechanism is a molded-in ratchet.

15. The assembly of claim 13, further comprising an elbow component having a fluid passageway there through and adapted to receive the irrigation tube when positioned within the assembly.

16. A speculum assembly for single-use in colon lavage therapy comprising:
a first tubular body having a tapered sleeve comprising a tip, a scallop opening, an annular rib about the first tubular body, and a reduced diameter band; and
a second tubular body to which the first tubular body is assembled, the second tubular body has a leading end, a middle portion and a trailing end; the leading end is complimentary to the first tubular body; the middle portion incorporates a mechanism that corresponds with an adjustable positioner assembled thereon, enabling positional adjustment along the second tubular body, the adjustable positioner incorporates at least one flexible tab; and the trailing end comprises a handle, the handle is adapted to accept a portion of an irrigation tube and a portion of a waste hose.

17. The assembly of claim 16, wherein irrigation fluid is delivered by the irrigation tube to a substantially forward portion of the assembly.

18. A speculum assembly for single-use in colon lavage therapy comprising:
a first tubular tapered sleeve comprising a tip, a scallop opening, and a flange, the flange has a snap-groove incorporated therein; and
a second tubular body is snap-fitted to the snap-groove of the first tubular body, the second tubular body is complimentary to the first tubular body having an annular snap-ring about a middle portion and comprises a handle, the handle is adapted to accept a portion of an irrigation tube and a portion of a waste hose.

19. The assembly of claim 18, wherein the first and the second tubular body may be radially adjusted during lavage therapy to obscure the scallop opening and flush of the first and second tubular body.
